(19) United States
(12) Patent Application Publication
(10) Pub. No.: US 2010/0286513 A1
(43) Pub. Date: Nov. 11, 2010

(54) Plunger Adapter for Coaxial Syringe System

(76) Inventors: Ralph E. Pollard, JR., Fairfield, OH (US); William A. Hagen, Canton, GA (US)

Correspondence Address: Mallinckrodt Inc., 675 McDonnell Boulevard, HAZELWOOD, MO, 63042, US

(21) Appl. No.: 12/863,449

(22) PCT Filed: Jan. 21, 2009
(86) PCT No.: PCT/US09/31473

§ 371 Date: Jul. 19, 2010

Related U.S. Application Data
(60) Provisional application No. 61/022,855, filed on Jan. 23, 2008.

(51) Int. Cl. A61B 6/00; A61M 5/31

(52) U.S. Cl. 600/432; 600/4

(57) ABSTRACT

A syringe assembly (10), is provided for sequentially injecting multiple medical fluids (16,68). The syringe assembly may include a first syringe (14) having a pushrod (22) coupled to a plunger head (20) disposed in a barrel (18). A front portion of the barrel may include a fitting (32). The syringe assembly may include an adapter (12) having a mating fitting (38) removably coupled to the fitting (32). The adapter (12) may include a plunger head (44) that has a flow control feature.

### RELATED APPLICATIONS

This application claims priority to U.S. Provisional Patent Application No. 61/022,855 entitled “PLUNGER ADAPTER FOR COAXIAL SYRINGE SYSTEM” filed on 23 Jan. 2008.

### FIELD OF THE INVENTION

The present invention relates generally to syringes and, more particularly, to syringe assemblies used for multiple sequential injections.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Nuclear medicine utilizes radioactive material for diagnostic and therapeutic purposes by injecting a patient with a small dose of the radioactive material, which tends to concentrate in certain organs or biological regions of the patient. Radioactive materials typically used for nuclear medicine include Technetium-99m, Indium-113m, and Strontium-87m among others. Some radioactive materials naturally concentrate toward a particular tissue, for example, iodine concentrates toward the thyroid. However, radioactive materials are often combined with a tagging or organ-seeking agent, which targets the radioactive material for the desired organ or biologic region of the patient. These radioactive materials alone or in combination with a tagging agent are typically referred to as radiopharmaceuticals in the field of nuclear medicine. At relatively lower doses of the radiopharmaceutical, a radiation imaging system (e.g., a gamma camera) provides an image of the organ or biological region that collects the radiopharmaceutical. Irregularities in the image are often indicative of a pathologic condition, such as cancer. Higher doses of the radiopharmaceutical may be used to deliver a therapeutic dose of radiation directly to the pathologic tissue, such as cancer cells.

In certain applications, multiple independent syringes are used to inject various substances, such as radiopharmaceuticals, tagging or organ seeking agents, contrast agents, biocompatible flushing substances, and so forth. The abundance and low cost of these syringes is very attractive for medical institutions, particularly in high volume areas involving disposal of the syringes. Unfortunately, the use of multiple independent syringes may result in delays, poor timing, and/or other problems with administering the multiple injections.

### SUMMARY

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

A first aspect of the present invention is directed to a syringe assembly for sequentially injecting multiple medical fluids. The syringe assembly includes a syringe having a pushrod coupled to a plunger head that is disposed in a barrel of the syringe. A front portion of the barrel of the syringe includes a fitting (e.g., a luer fitting) to which an adapter having a mating fitting (e.g., a luer fitting) is removably coupled. This adapter includes a plunger head that has a flow control feature. The flow control feature may be defined as a structure or mechanism that controls and/or enables a user to control the flow of a fluid between two or more portions (e.g., opposite sides) of the flow control feature. For example, the flow control feature may enable and/or disable flow of fluid through and/or around the plunger head in response to an input, pressure differential, and/or movement of parts. For example, in some embodiments, the flow control feature may be closed or block flow of fluid until the input, pressure differential, and/or movement of parts changes the state of the flow control feature to an open position. For example, the flow control feature may include a check valve, a membrane that can be pierced, or a combination thereof.

A second aspect of the invention is directed to an adapter for converting syringes into a multi-injection syringe assembly (e.g., coaxial barrels, plungers, etc.). The adapter includes a plunger head having a through passage with a flow control feature. The plunger head includes a fitting configured to connect with a mating fitting disposed on a front portion of a syringe barrel. The through passage of the plunger head may be defined as a passage that extends through the plunger head from one side to another. For example, the through passage may extend completely through the plunger head in an axial direction relative to a longitudinal axis of the syringe assembly, such that the through passage is open to both upstream and downstream sides of the plunger head. In some embodiments, the through passage may be disposed directly along the axis of the plunger head. The through passage may have a variety of geometries, such as a cylindrical passage.

Yet a third aspect of the invention is directed to a method of using a syringe assembly. In this method, a plunger head is removably connected to a front portion of a syringe barrel. This plunger head includes a through passage having a flow control feature.

Still fourth aspect of the invention is directed to a method of using a syringe assembly. In this method, a first syringe barrel and a removable plunger head that is removably coupled thereto are moved lengthwise along an interior of a second syringe barrel toward a front portion of the second syringe barrel. While the first syringe barrel and the removable plunger head are moved, a flow control feature in the removable plunger head remains closed.

Numerous refinements exist of the features noted above in relation to the various aspects of the present invention. Further features may also be incorporated in these various aspects as well. These refinements and additional features may exist individually or in any combination. For instance, various features discussed below in relation to one or more of the illustrated embodiments may be incorporated into any of the above-described aspects of the present invention alone or in any combination. Again, the brief summary presented above is intended only to familiarize the reader with certain aspects and contexts of the present invention without limitation to the claimed subject matter.

### BRIEF DESCRIPTION OF THE FIGURES

Various features, aspects, and advantages of the present invention will become better understood when the following detailed description is read with reference to the accompanying figures in which like characters represent like parts throughout the figures, wherein:

FIG. 1 is an exploded perspective view of an exemplary syringe assembly;

FIG. 2 is a cross-section of the syringe assembly in a first injection stage;

FIG. 3 is a cross-section of the syringe assembly in a second injection stage;

FIGS. 4 and 5 are front and rear perspective views of an exemplary plunger adapter of the syringe assembly;

FIG. 6 is a front view of the plunger adapter;

FIG. 7 is a cross-section of the plunger adapter;

FIG. 8 is an exploded perspective view of an alternative embodiment of a syringe assembly;

FIG. 9 is a cross-section of the alternative syringe assembly in a first injection stage;

FIG. 10 is a cross-section of the alternative syringe assembly in a second injection stage;

FIG. 11 is a cross-section of a plunger adapter of the alternative syringe assembly;

FIG. 12 is a flow chart of a nuclear medicine process;

FIG. 13 is a diagram of a system for loading a syringe with a radioisotope; and

FIG. 14 is a diagram of a nuclear imaging system.

### DETAILED DESCRIPTION OF SPECIFIC EMBODIMENTS

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a”, “an”, “the”, and “said” are intended to mean that there are one or more of the elements. The terms “comprising”, “including”, and “having” are intended to be inclusive and mean that there may be additional elements other than the listed elements. Moreover, the use of “top”, “bottom”, “above”, “below” and variations of these terms is made for convenience, but does not require any particular orientation of the components. As used herein, the term “coupled” refers to two or more objects being in direct contact with one another or being directly or indirectly connected. Additionally, the phrase “in fluid communication” or “fluidly coupled” indicates that fluid and/or fluid pressure may be transmitted from one object to another. Similarly, the phrase “in thermal communication” or “thermally coupled” indicates that heat may be transferred from one object to another.

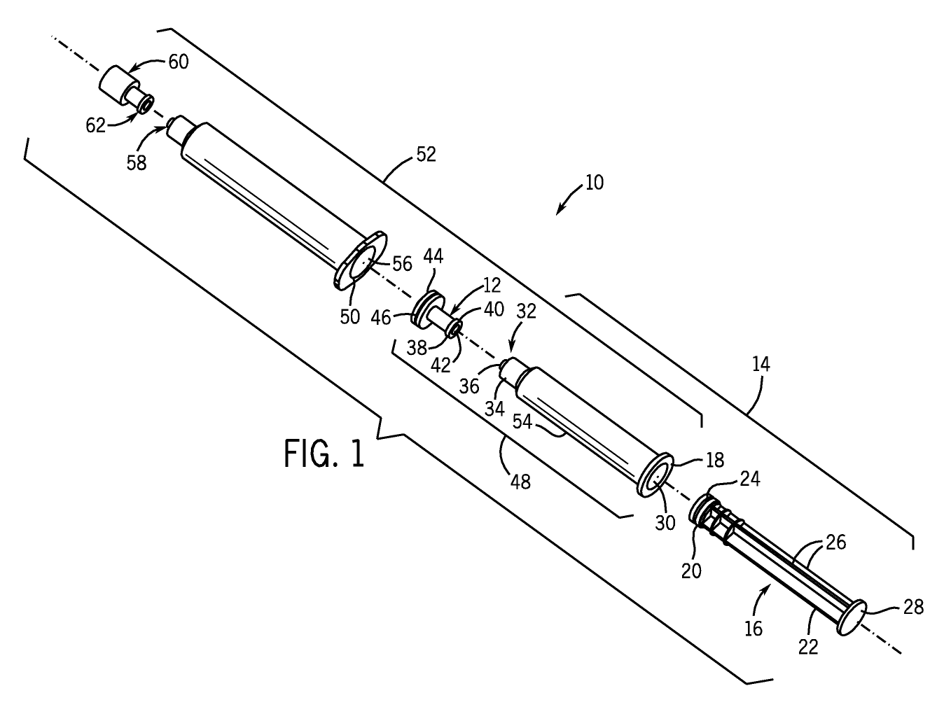

FIG. 1 is an exploded perspective view of an exemplary syringe assembly 10 having a unique adapter 12 configured to serve as a plunger head to convert two conventional syringes into a coaxial arrangement having one syringe concentrically inside the other for sequential injection of two different fluids. As discussed in detail below, the adapter 12 may include a fitting, such as a luer fitting 38, for quick attachment to a mating fitting, such as luer fitting 32, disposed on a syringe barrel 18. The adapter 12 may include a plunger head 44 with a suitable flow control feature, such as a check valve, one or more thin membranes, or a combination thereof. Once coupled, the adapter 12 combined with the barrel 18 may serve as a plunger 48 for another larger barrel 50, thereby enabling sequential injection of one fluid after another in a simple and low cost manner as discussed in detail below. In other words, rather than a complex and costly multi-barrel syringe design, the disclosed adapter 12 is a simple, low cost, and quick converter to combine readily available syringe components, e.g., different sized syringe barrels 18 and 50. Moreover, the adapter 12 may be provided in multiple diameters to act as a converter for a broad range of syringe barrels.

Specifically, the illustrated syringe assembly 10 includes a first syringe 14 having a first plunger 16 that extends into a first barrel 18. The first plunger 16 includes a first plunger head 20 coupled to a first push rod 22. The first plunger head 20 may be formed of a rubber material and may have one or more O-rings 24 to create a seal as the first plunger 16 moves along the interior of the first barrel 18. The first push rod 22 may include a plurality of ribs 26 and a thumb tab 28. The first barrel 18 may have a generally cylindrical shape with an open end 30 to receive the first plunger 16 and a luer fitting 32 configured to couple with the adapter 12. Specifically, the illustrated luer fitting 32 may include a collar 34 disposed about a male luer 36. Thus, the illustrated adapter 12 may include a substantially complimentary luer fitting 38 with a flange 40 disposed about a female luer 42. As a result, the luer fittings 32 and 38 may be removably coupled together, such that the adapter 12 can be installed and removed with ease from the first barrel 18 of the first syringe 14. In addition, the adapter 12 may include a second plunger head 44, which may be made of a rubber material and may include one or more O-rings 46.

In the illustrated embodiment of FIG. 1, the first barrel 18 may be combined with the adapter 12 to form a second plunger 48 that may be inserted within a second barrel 50 to define a second syringe 52. In other words, the first barrel 18 of the first syringe 14 may serve as a second push rod 54 that combines with the second plunger head 44 to define the second plunger 48. Thus, the first barrel 18 and the second push rod 54 are the same element in the illustrated embodiment, and serve as a common element between the first syringe 14 and the second syringe 52. In addition, the adapter 12 may serve as the linking member in between the first and second syringes 14 and 52 by converting the first barrel 18 into the second push rod 54 of the second plunger 48. Again, the second plunger 48 is configured to slide lengthwise through the second barrel 50. The second barrel 50 may have a generally cylindrical shape with an open end 56 to receive the second plunger head 44. The second barrel 50 may have a luer fitting 58 configured to mate with a luer fitting of various attachments. For example, in the illustrated embodiment, a plug 60 has a luer fitting 62 configured to mate with the luer fitting 58 disposed on the second barrel 50. The plug 60 may be used to seal the second barrel 50 after filling it with a fluid, such as a medical fluid. The first plunger 16 may be inserted into the first barrel 18 to seal a fluid, such as another medical fluid, within the first syringe 14. The syringe assembly 10 may be filled and shipped to a customer as a pre-filled syringe ready for use.

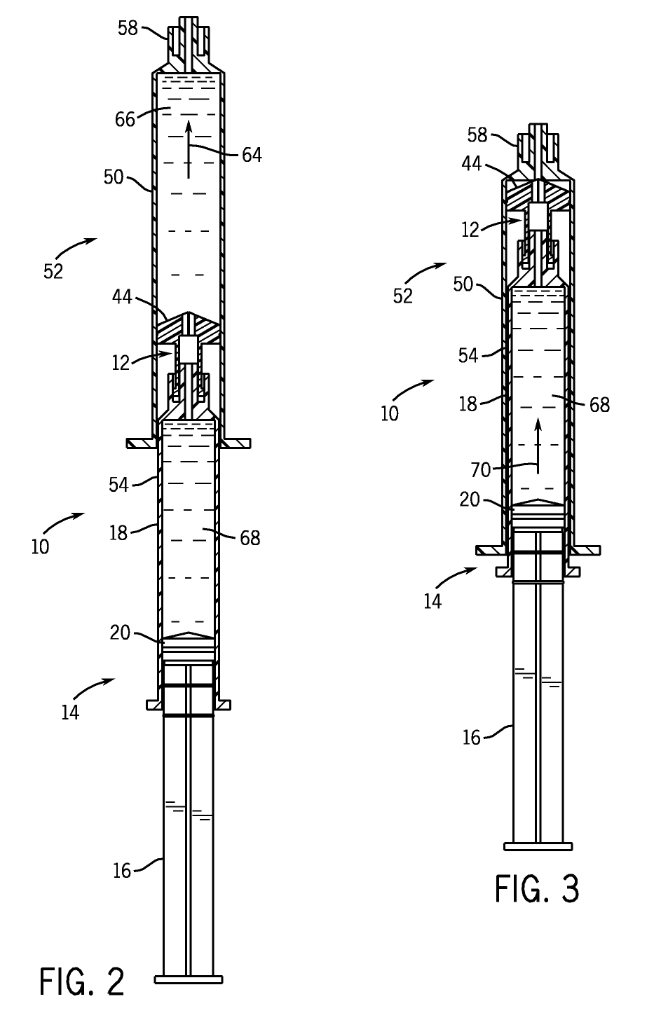

FIG. 2 is a cross-sectional view of the syringe assembly 10 illustrating a first injection stage 64 of a fluid 66 disposed within the second barrel 50 of the second syringe 52. Specifically, the first barrel 18 may serve as the second push rod 54 to force the second plunger head 44 of the adapter 12 lengthwise through the second barrel 50, thereby forcing the fluid 66 to exit out through the luer fitting 58 as indicated by arrow 64 of the first injection stage. During this motion, the second plunger head 44 may act as a check valve to generally block flow of a fluid 68 disposed in the first barrel 18. In other words, if a user pushes on the first push rod 22, then the fluid 68 disposed within the first barrel 18 may remain contained within the first barrel 18 due to the check valve function of the second plunger head 44. The check valve function of the second plunger head 44 may continue until sufficient pressure of the fluid 68 overcomes a corresponding pressure of the fluid 66. The fluids 66 and 68 may include a variety of medical fluids. For example, the fluid 66 may include a radiopharmaceutical, a contrast agent, a drug, or a combination thereof. By further example, the fluid 68 may include a biocompatible flushing or cleaning substance, such as a heparin solution, sterilized water, a glucose solution, saline, or another suitable substance.

FIG. 3 is a cross-sectional view of the syringe assembly 10 illustrating a second injection stage 70 of the fluid 68 disposed within the first barrel 18 of the first syringe 14. Specifically, upon reaching the front of the second barrel 50, the check valve function of the second plunger head 44 may open up fluid flow to enable the fluid 68 to pass out through the luer fitting 58 of the second barrel 50. In other words, the second plunger head 44 can no longer move when abutted against the front of the second barrel 50, such that the pressure of the fluid 68 within the first barrel 18 may overcome the check valve function of the second plunger head 44. As a result, the fluid 68 is no longer blocked by the check valve function of the second plunger head 44. As appreciated, the check valve function of the second plunger head 44 may embody a variety of different check valve features and configurations, such as resilient material that closes a passage until sufficient pressure moves the resilient material.

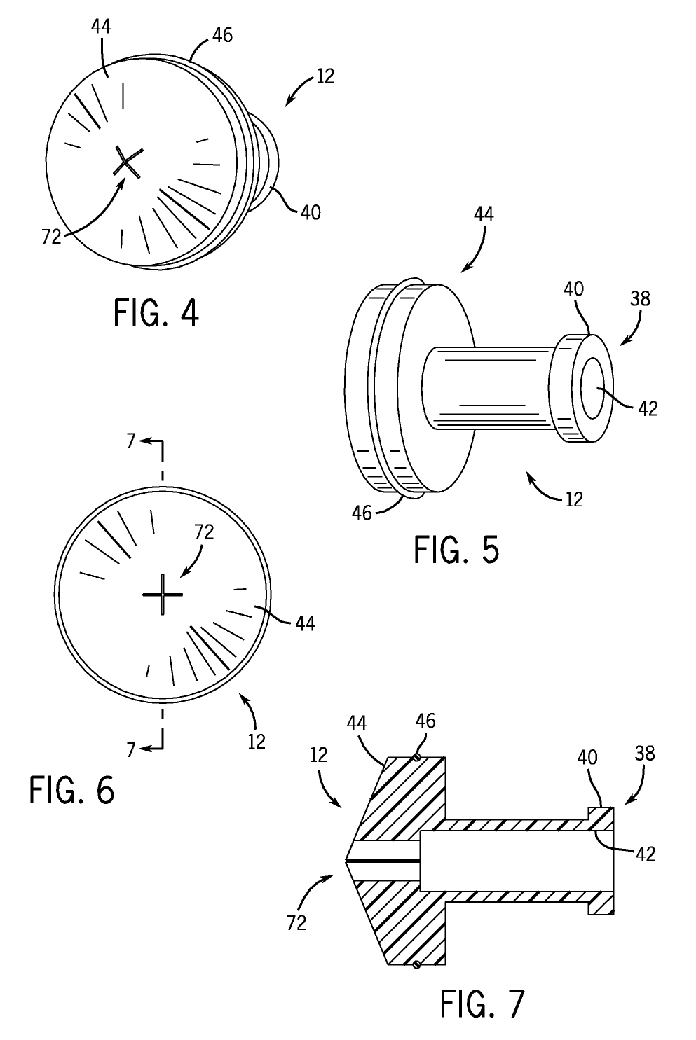

FIGS. 4, 5, 6, and 7 illustrate one exemplary embodiment of the adapter 12 having a check valve 72 disposed in the second plunger head 44. With reference to all four of these figures, the check valve 72 may have a cross-shaped passage 74 that extends through the second plunger head 44 to the female luer 42. In other words, the passage 74 may be formed by a plurality of slits or thin flat passages that intersect with one another. In the illustrated embodiment, the second plunger head 44 is made of an elastic or resilient material, such as rubber, such that the cross-shaped passage 74 of the check valve 72 remains normally closed until sufficient pressure acts on the passage 74. At that time, the pressure overcomes the resilient nature of the material keeping the cross-shaped passage 74 closed, and results in the cross-shaped passage 74 opening up to permit fluid passage therethrough. Other embodiments of the check valve 72 may exhibit other appropriate check valve designs/configurations within the scope of the disclosed embodiments.

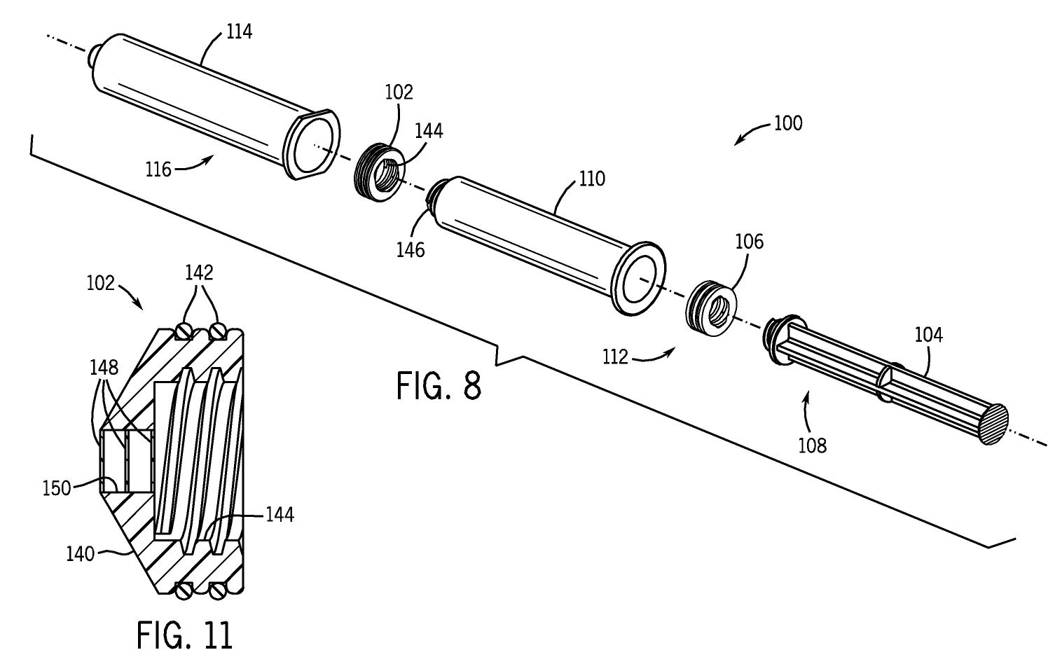

The check valve 72 may have a thin membrane that seals the passage 74 until a spike within the second barrel 50 pierces through the membrane. An embodiment such as this is illustrated and described below with reference to FIGS. 8-11.

FIG. 8 shows an alternative embodiment of a syringe assembly 100 having an alternative plunger adapter 102 with a thin membrane as discussed in further detail below. Specifically, the illustrated syringe assembly 100 may include a push rod 104 configured to couple with a plunger head 106 to define a plunger 108. The plunger 108 may be inserted within a barrel 110 to form a syringe 112 similar to the syringe 14 in the embodiment described above with reference to FIG. 1. In turn, the plunger adapter 102 may be coupled with the barrel 110 of the syringe 112 to serve as a plunger for insertion into a barrel 114. Again, similar to the syringe 52 in the embodiment of FIG. 1, another syringe 116 may be formed by the combination of the barrel 114 with the plunger adapter 102 coupled to the barrel 110 sliding lengthwise through the barrel 114. The syringe assembly 110 may operate similar to the syringe assembly 10 as illustrated and described above with reference to FIGS. 1-3 with the exception of the flow control feature of the plunger adapter 102.

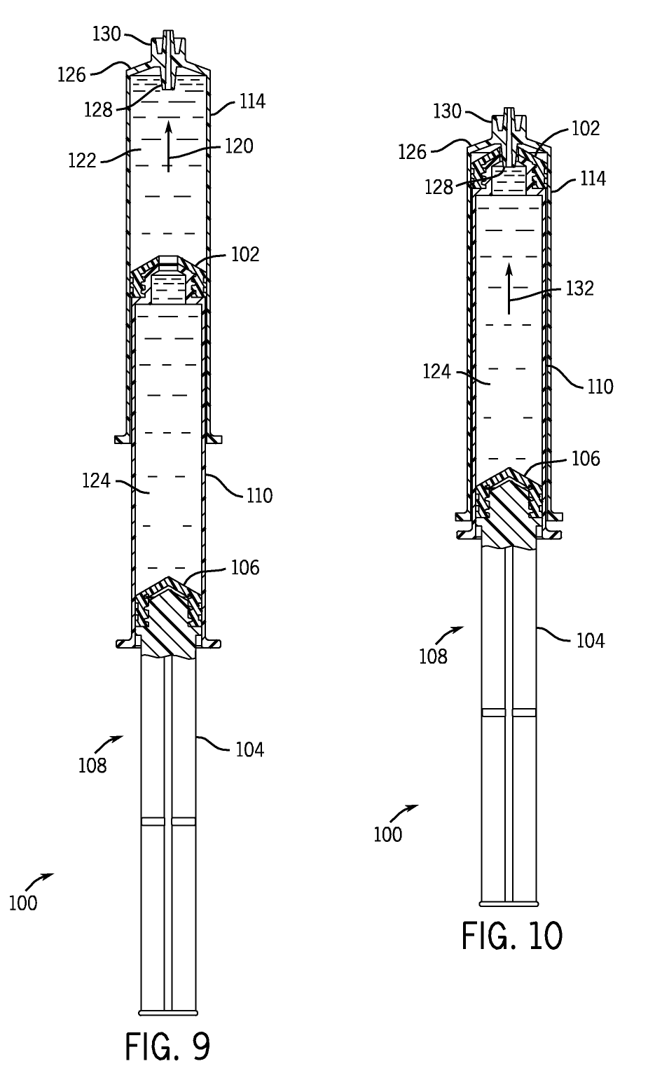

FIG. 9 is a cross-sectional view of the syringe assembly 100 illustrating a first injection stage 120 of a fluid 122 disposed within the barrel 114 of the syringe 116. As illustrated, the plunger adapter 102 coupled to the barrel 110 serves both to move the fluid 22 lengthwise through the barrel 114, while also blocking flow of a fluid 124 disposed within the barrel 110 of the syringe 112 during such movement. In this exemplary embodiment, the plunger adapter 102 includes a thin membrane that may completely block flow of the fluid 124 or the fluid 122 through the plunger adapter 102. However, at a front portion 126 of the barrel 114, a hollow spike 128 may be arranged opposite from a luer fitting 130 to pierce the thin membrane disposed within the plunger adapter 102 upon reaching the front portion 126. FIG. 10 is a cross-sectional view of the syringe assembly 100 illustrating a second injection stage 132, wherein the fluid 124 flows out through the plunger adapter 102 and the luer fitting 130. Specifically, the hollow spike 128 may pierce the thin membrane disposed within the plunger adapter 102, thereby opening flow of the fluid 124.

FIG. 11 is a cross-sectional view of an exemplary embodiment of the plunger adapter 102 of FIGS. 8, 9, and 10. Specifically, the illustrated plunger adapter 102 may include a plunger head 140 having one or more O-rings 142 and internal threads 144 configured to couple with threads 146 disposed on the barrel 110. The plunger head 140 may be made of a suitable plastic, rubber, or metal, while the O-rings 142 may be made of rubber or another resilient material. The plunger adapter 102 may include one or more thin membranes 148 disposed across a passage 150 in the plunger head 140. These thin membranes 148 generally seal off the passage 150 until the hollow spike 128 within the barrel 114 pierces the membranes 148. In certain embodiments, the thin membranes 148 may be made of a thin sheet of plastic, metal, or another suitable material. Moreover, the thin membranes 148 may be part of an insert that is sealed within the passage 150. However, as discussed above, the one or more thin membranes 148 serve to block fluid flow through the plunger head 140 until being pierced by the hollow spike 128.

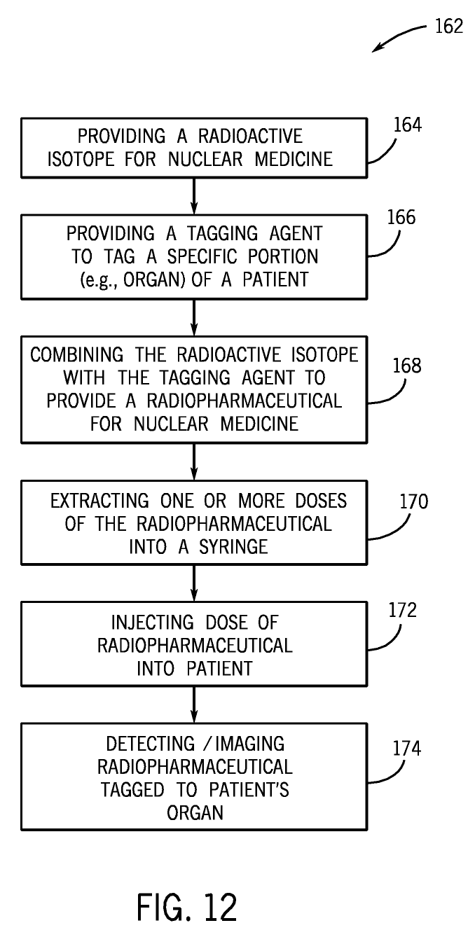

FIG. 12 is a flowchart illustrating an exemplary nuclear medicine process that uses the syringe assemblies 10 and 100 discussed in detail above. As illustrated, the process 162 begins by providing a radioactive isotope for nuclear medicine at block 164. For example, block 164 may include eluting technetium-99m from a radioisotope generator. At block 166, the process 162 proceeds by providing a tagging agent (e.g., an epitope or other appropriate biological directing moiety) adapted to target the radioisotope for a specific portion (e.g., an organ) of a patient. At block 168, the process 162 then proceeds by combining the radioactive isotope with the tagging agent to provide a radiopharmaceutical for nuclear medicine. In certain embodiments, the radioactive isotope may have natural tendencies to concentrate toward a particular organ or tissue and, thus, the radioactive isotope may be characterized as a radiopharmaceutical without adding any supplemental tagging agent. At block 170, the process 162 then may proceed by extracting one or more doses of the radiopharmaceutical into a syringe or another container, such as the disclosed syringe assemblies 10 and 100, suitable for administering the radiopharmaceutical to a patient in a nuclear medicine facility or hospital. The syringe assemblies 10 and 100 may be filled with a flushing agent for the second injection stage as discussed in detail above. At block 172, the process 162 proceeds by injecting or generally administering a dose of the radiopharmaceutical into a patient. Again, the process 162 may include the second injection stage to flush out the syringe assemblies 10 and 100, as well as any associated tubing that is connected to the syringe assembly. After a pre-selected time, the process 162 proceeds by detecting/imaging the radiopharmaceutical tagged to the patient's organ or tissue (block 174). For example, block 174 may include using a gamma camera or other radiographic imaging device to detect the radiopharmaceutical disposed on or in or bound to tissue of a brain, a heart, a liver, a tumor, a cancerous tissue, or various other organs or diseased tissue.

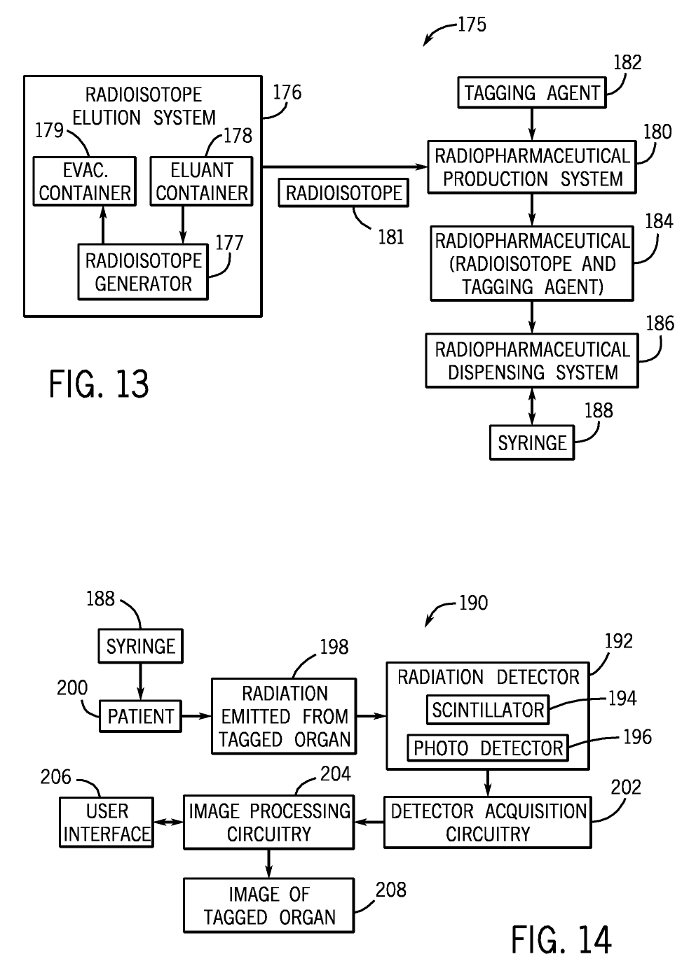

FIG. 13 is a block diagram of an exemplary system 175 for providing a syringe having a radiopharmaceutical disposed therein for use in a nuclear medicine application. As illustrated, the system 175 includes a radioisotope elution system 176 having a radioisotope generator 177 configured to receive an eluant (e.g., a saline solution) from an eluant container 178 and output an eluate (e.g., daughter radioisotope) to an evacuated container 179. The system 175 also includes a radiopharmaceutical production system 180, which functions to combine a radioisotope 181 (e.g., technetium-99m solution acquired through use of the radioisotope elution system 176) with a tagging agent 182. In some embodiment, this radiopharmaceutical production system 180 may refer to or include what are known in the art as “kits” (e.g., Technescan® kit for preparation of a diagnostic radiopharmaceutical). Again, the tagging agent may include a variety of substances that are attracted to or targeted for a particular portion (e.g., organ, tissue, tumor, cancer, etc.) of the patient. As a result, the radiopharmaceutical production system 180 produces or may be utilized to produce a radiopharmaceutical including the radioisotope 181 and the tagging agent 182, as indicated by block 184. The illustrated system 175 may include a radiopharmaceutical dispensing system 186, which facilitates extraction of the radiopharmaceutical into a vial or syringe 188 (e.g., a syringe assembly 10 or 100 of FIGS. 1-11). In certain embodiments, the various components and functions of the system 175 are disposed within a radiopharmacy, which prepares the syringe 188 of the radiopharmaceutical for use in a nuclear medicine application. For example, the syringe 188 may be prepared and delivered to a medical facility for use in diagnosis or treatment of a patient.

FIG. 14 is a block diagram of an exemplary nuclear medicine imaging system 190 utilizing the syringe 188 of radiopharmaceutical provided using the system 176 of FIG. 13. As illustrated, the nuclear medicine imagining system 190 includes a radiation detector 192 having a scintillator 194 and a photo detector 196. In response to radiation 198 emitted from a tagged organ within a patient 200, the scintillator 194 emits light that is sensed and converted to electronic signals by the photo detector 196. Although not illustrated, the imaging system 190 can include a collimator to collimate the radiation 198 directed toward the radiation detector 192. The illustrated imaging system 190 includes detector acquisition circuitry 202 and image processing circuitry 204. The detector acquisition circuitry 202 generally controls the acquisition of electronic signals from the radiation detector 192. The image processing circuitry 204 may be employed to process the electronic signals, execute examination protocols, and so forth. The illustrated imaging system 190 includes a user interface 206 to facilitate user interaction with the image processing circuitry 204 and other components of the imaging system 190. As a result, the imaging system 190 produces an image 208 of the tagged organ within the patient 200. Again, the foregoing procedures and resulting image 208 directly benefit from use of the syringe assemblies 10 and 100.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cap all modifications, equivalents, and alternatives falling within the spirit and scope of the invention as defined by the following appended claims.

1-31. (canceled)

32. A syringe assembly for sequentially injecting multiple medical fluids, comprising:
a first syringe comprising a first pushrod coupled to a first plunger head that is disposed in a first barrel, wherein a front portion of the first barrel comprises a luer fitting; and
an adapter comprising a mating luer fitting removably coupled to the luer fitting of the first barrel, wherein the adapter comprises a second plunger head having a flow control feature.

33. The assembly of claim 32, further comprising:
a second syringe having a second barrel, wherein the second plunger head of the adapter is disposed within the second barrel of the second syringe.

34. The assembly of claim 33, wherein the first barrel is configured to function as a second pushrod to move the second plunger head inside the second barrel.

35. The assembly of claim 33, further comprising:
one or more medical fluids disposed in the first barrel, or the second barrel, or a combination thereof.

36. The assembly of claim 32, wherein the flow control feature comprises a check valve.

37. The assembly of claim 32, wherein the flow control feature comprises a passage extending through a resilient portion of the second plunger head.

38. The assembly of claim 37, wherein the passage comprises a cross-shaped passage.

39. The assembly of claim 32, wherein the flow control feature comprises a membrane.

40. The assembly of claim 39, wherein a front portion of the second barrel comprises a spike configured to pierce the membrane.

41. An adapter for converting syringes into a multi-injection syringe assembly, comprising:
a plunger head comprising a through passage having a flow control feature, wherein the plunger head comprises a luer fitting configured to connect with a mating luer fitting of a syringe barrel.

42. The adapter of claim 41, wherein the flow control feature comprises a check valve.

43. The adapter of claim 42, wherein the check valve comprises a resilient portion of the plunger head disposed about the through passage.

44. The adapter of claim 43, wherein the through passage comprises a plurality of slits extending through the resilient portion of the plunger head.

45. The adapter of claim 41, wherein the flow control feature comprises a membrane disposed across the through passage.

46. A method of using a syringe assembly, comprising:
removably connecting a plunger head to a front portion of a syringe barrel, wherein the plunger head comprises a through passage having a flow control feature, and the removably connecting comprises engaging a luer fitting of the plunger head with a mating luer fitting of the front portion of the syringe barrel.

47. The method of claim 46, further comprising:
inserting the plunger head that is coupled to the syringe barrel into another syringe barrel such that the syringe barrels are in a coaxial arrangement.

48. The method of claim 47, further comprising:
filling the syringe barrel with a medical fluid, or filling the another syringe barrel with another medical fluid, or a combination thereof.

49. The method of claim 48, further comprising:
inserting a plunger into the syringe barrel, or coupling a plug to a front portion of the another syringe barrel, or a combination thereof.

50. The method of claim 47, wherein the flow control feature remains closed during movement of the syringe barrel along a length of the another syringe barrel until the plunger head reaches a front portion of the another syringe barrel.

51. The method of claim 50, wherein the flow control feature comprises a membrane, and the another syringe barrel comprises a spike to pierce the membrane at the front portion of the another syringe barrel.
